(19) United States
(12) Patent Application Publication
(10) Pub. No.: US 2016/0310626 A1
(43) Pub. Date: Oct. 27, 2016

(54) REMOVABLE HOLDER APPARATUS

(71) Applicant: JULIUS SÄMANN LTD., Hamilton (BM)

(72) Inventors: Gino DUCA, Jamesville, NY (US); Michael Joseph GRIEB, Marcellus, NY (US); Michael B. CRAWFORD, Sr., Morrisville, NY (US); Beth Ann ARTHUR, Sackets Harbor, NY (US); Scott Jacob GOODRICH, Sackets Harbor, NY (US); Daniel SÄMANN, Watertown, NY (US)

(21) Appl. No.: 15/139,152

(22) Filed: Apr. 26, 2016

Related U.S. Application Data
(60) Provisional application No. 62/153,188, filed on Apr. 27, 2015.

(51) Int. Cl. A61L 9/12

(52) U.S. Cl. CPC A61L 9/12; A61L 2209/15

(57) ABSTRACT

A holder apparatus for air fresheners may include, for example, a base having a front side and a back side; a front-side holding mechanism; and a back-side mounting mechanism. The front-side holding mechanism may be configured to hold an air freshener in a manner such that the air freshener is exposed on at least one side in an amount greater than about 50% of the surface area of the at least one side. The back-side mounting mechanism may be configured to mount the base onto a mounting surface.

### CLAIM OF PRIORITY UNDER 35 U.S.C. §119

The present application for patent claims the benefit of U.S. Provisional Application No. 62/153,188, entitled “Removable Holder Apparatus,” filed Apr. 27, 2015, which is expressly incorporated herein by reference in its entirety.

### BACKGROUND

1. Field

The present disclosure relates generally to air fresheners, and more particularly to air freshener holders and the like.

2. Background

Air fresheners typically emit a fragrance or deodorize the air in an enclosed space. They are often used for household or commercial purposes such as for pleasant odor or sanitizing the air. For example, air fresheners are often used in a car, a hotel lobby, an office lobby, a living room, a bedroom, a hospital room, or a restroom.

Air fresheners are typically made from a combination of scented oils, a container, and a delivery mechanism. Example containers include wax, capsules, jars, and so on. Example delivery mechanisms include heating elements, plug-in systems, sticks, aerosols, and so on. For instance, scented oil can be mixed with wax to create a scented candle. When the scented candle is heated, the melting wax releases the scented oil into the air in the form of fumes. In another example, the scented oil can be packed in a container such as an oil capsule and the oil can be delivered using a plug-in system in which the oil capsule is plugged into an adapter that electrically heats the oil in the oil capsule, thus releasing the scented oil fumes into the air. In another example, a jar containing oil-infused sticks may be used. In another example, a spray-type mechanism may be used to deliver a scented aerosol material into the air.

Air fresheners used in vehicles are typically divided into two major categories: (i) hanging-type air fresheners and (ii) direct mount-type air fresheners that mount typically on an air vent. Hanging-type air fresheners are usually formed of a blotter stock that is impregnated with a fragrance oil-based composition. Direct mount-type air fresheners are often formed with the fragrance oil-based composition contained within a container, typically a capsule, with fixed mounting support.

While different air fresheners can be hanged from vehicular surfaces, mounted on an air vent, or placed in a cup holder, the mounting location is limited by the type of mounting attachment and the delivery mechanism. For example, a hanging-type air freshener typically needs a hanging attachment, such as a string forming a loop and running through a hole in the air freshener itself, and is typically hung from its hanging attachment. A direct mount-type air freshener using air flow from an air conditioning system as the delivery mechanism is typically attached with a vent clip, which may not be mounted on a window or the side of a dashboard. Further, the mounting generally cannot be reused since it is fixed to the air freshener. Still further, if the air freshener is detached from the mounting mechanism and placed on a surface, the oil in the air freshener may leave a mark on the surface and even damage the surface finish. Placing the air freshener in a cup holder occupies the cup holding space and can be inconvenient when passengers need to use the cup holder for beverages or other items.

There accordingly remains a need for improved air fresheners and flexible mounting arrangements.

### SUMMARY

Embodiments disclosed herein address the above-stated needs by providing improved air fresheners and flexible mounting arrangements, as well as methods of making and using the same.

As an example, a holder apparatus for air fresheners is provided herein that includes a base having a front side and a back side; a front-side holding mechanism; and a back-side mounting mechanism. The front-side holding mechanism may be configured to hold an air freshener in a manner such that the air freshener is exposed on at least one side in an amount greater than about 50% of the surface area of the at least one side. The back-side mounting mechanism may be configured to mount the base onto a mounting surface.

The holding mechanism may include, for example, a holding frame protruding from the front side of the base and arranged to at least partially encircle the air freshener. The holding frame may have an inside surface and an outside surface, with the inside surface including one or more notches arranged to hold the air freshener within the holding frame. The one or more notches may include a plurality of notches spaced apart from each other along the inside surface of the holding frame.

In addition or as an alternative, the holding mechanism may include a latch configured to hold the air freshener to the front side of the base. The holding mechanism may also include an adhesive strip or layer arranged to hold the air freshener to the front side of the base. The holding mechanism may also include a hook-and-loop-based connector arranged to hold the air freshener to the front side of the base. The holding mechanism may also include a tack arranged to hold the air freshener to the front side of the base.

For its part, the mounting mechanism may include, for example, a skid-resistant material arranged to mount the base onto the mounting surface. The mounting mechanism may also include a hook-and-loop-based connector arranged to mount the base onto the mounting surface. The mounting mechanism may also include an adhesive strip or layer arranged to mount the base onto the mounting surface. The mounting mechanism may also include a suction cup connector arranged to mount the base onto the mounting surface. The mounting mechanism may also include a magnetic material arranged to mount the base onto the mounting surface.

In some designs, the holder apparatus may further include one or more grooves recessed into the front side of the base. In addition or as an alternative, the holder apparatus may further include one or more knock-out openings penetrating the front side and back side of the base.

The base may be made of a non-metal material, a polymeric material, or other materials.

An air freshener assembly is also provided that includes such a holder apparatus and an air freshener held by the holder apparatus.

A holder apparatus for air fresheners is also provided that includes a base having a front side and a back side; front-side holding means; and back-side holding means. The front-side holding means may be for holding an air freshener in a manner such that the air freshener is exposed on at least one side in an amount greater than about 50% of the surface area of the at least one side. The back-side mounting means may be for mounting the base onto a mounting surface.

A method of deploying an air freshener assembly is also provided. The method may include, for example, providing a holder apparatus including a base having a front side and a back side, a front-side holding mechanism, and a back-side mounting mechanism; connecting an air freshener to the front-side holding mechanism in a manner such that the air freshener is exposed on at least one side in an amount greater than about 50% of the surface area of the at least one side; and mounting the back-side mounting mechanism onto a mounting surface

Various aspects and features of the disclosure are described in further detail below with reference to various examples thereof as shown in the accompanying drawings. While the present disclosure is described below with reference to various examples, it should be understood that the present disclosure is not limited thereto. Those of ordinary skill in the art having access to the teachings herein will recognize additional implementations, modifications, and examples, as well as other fields of use, which are within the scope of the present disclosure as described herein, and with respect to which the present disclosure may be of significant utility.

### BRIEF DESCRIPTION OF THE DRAWINGS

The accompanying drawings are presented to aid in the description of embodiments of the invention and are provided solely for illustration of the embodiments and not limitation thereof.

FIG. 1 is a perspective view of an example air freshener assembly.

FIGS. 2A-2C illustrate, respectively, a perspective view, an end view, and a front view of a holder apparatus implemented with an example holding mechanism.

FIG. 3 is a front perspective view of a holder apparatus implemented with another example holding mechanism.

FIG. 4 is a front perspective view of a holder apparatus implemented with another example holding mechanism.

FIG. 5 is a front perspective view of a holder apparatus implemented with another example holding mechanism

FIG. 6 is a front perspective view of a holder apparatus implemented with another example holding mechanism.

FIG. 7 is a back perspective view of a holder apparatus implemented with an example mounting mechanism.

FIG. 8 is a back perspective view of a holder apparatus implemented with another example mounting mechanism.

FIG. 9 is a back perspective view of a holder apparatus implemented with another example mounting mechanism.

FIG. 10 is a back perspective view of a holder apparatus implemented with another example mounting mechanism.

FIG. 11 is a back perspective view of a holder apparatus implemented with another example mounting mechanism.

FIG. 12 is a back perspective view of a holder apparatus implemented with example knock-out openings.

FIG. 13 is a flow diagram illustrating an example method of deploying an air freshener assembly.

### DETAILED DESCRIPTION

Aspects of the present invention are disclosed in the following description and related drawings directed to specific embodiments of the invention. The term “embodiments of the invention” does not require that all embodiments of the invention include the discussed feature, advantage, process, or mode of operation, and alternate embodiments may be devised without departing from the scope of the invention. Additionally, well-known elements of the invention may not be described in detail or may be omitted so as not to obscure other, more relevant details.

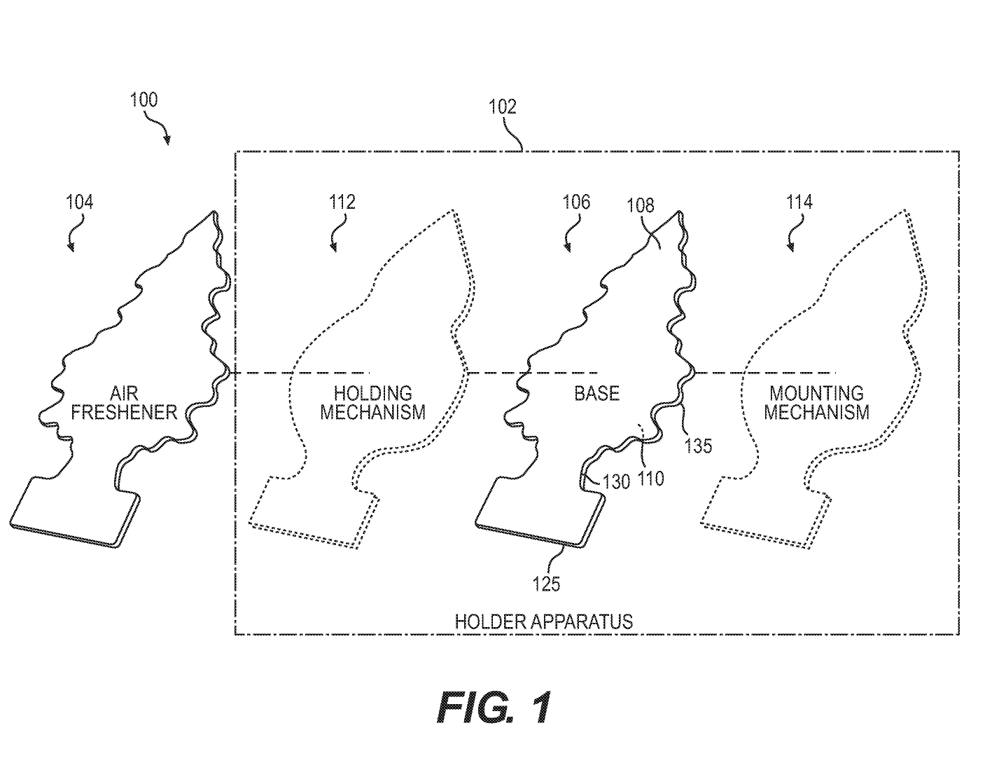

FIG. 1 is a perspective view of an example air freshener assembly. As shown, the air freshener assembly 100 is generally composed of a holder apparatus 102 and an air freshener 104 (e.g., a blotter stock air freshener, a wood air freshener, a polyvinyl chloride (PVC) air freshener, etc.). The holder apparatus 102 includes a base 106 having a front side 108 and a back side 110, a front-side holding mechanism 112, and a back-side mounting mechanism 114. The front-side holding mechanism 112 is configured to hold the air freshener 104 in a manner such that the air freshener 104 is exposed on at least one side in an amount greater than about 50% of the surface area of that side (preferably greater than about 60% of the surface area, more preferably greater than about 75% of the surface area, still more preferably greater than about 90% of the surface area, or most preferably greater than about 95% of the surface area). The back-side mounting mechanism 114 is configured to mount the base 106 onto a mounting surface (e.g., a surface of a desired object such as a windshield, dashboard, refrigerator, etc.).

In the illustrated example, the shape of the base 106, and the holder apparatus 102 generally, is divided into three parts, including a bottom region 125, a trunk region 130, and a crown region 135. It will be appreciated, however, that this design is shown for illustration purposes only, and that in general the holder apparatus 102 may take any shape as desired for a given application. Further, while the air freshener 104 as well as the holding mechanism 112 and the mounting mechanism 114 are shown as being commensurate in both shape and size as compared to the base 106, it will be appreciated that this is also for illustration purposes only, and that the air freshener 104, the holding mechanism 112, and the mounting mechanism 114 may vary in both shape and size in relation to the base 106 as desired for any given application.

The base 106 may be made of a variety of materials, including polymeric and other non-metal materials, or generally any material that is resistant to the fragrance oils used for the air freshener 104. For example, it may be made of a polymeric material such as a thermoplastic elastomer (like Santoprene), neoprene, a polyurethane material, or a polyolefin material, such as low or high density polyethylene or polypropylene. In some designs, the base 106 may be formed from a blend of polymeric materials, preferably a blend containing from about 25% to about 100% recycled plastics. The base 106 may be flexible to allow for insertion and removal of the air freshener 104 with ease, such as by flexing. The base 106 can be manufactured using any conventional manufacturing process such as thermo-forming, stamping, injection molding, vacuum forming, and so on.

The front-side holding mechanism 112 may be implemented in a variety of ways. For example, it may correspond to or otherwise include a holding frame to retain the air freshener 104 and secure it to the front side 108 of the base 106. In addition or as an alternative, it may correspond to or otherwise include a latch, an adhesive strip or layer, a hook-and-loop-based connector (e.g., VELCRO), a tack, and so on, or any combination of such connectors.

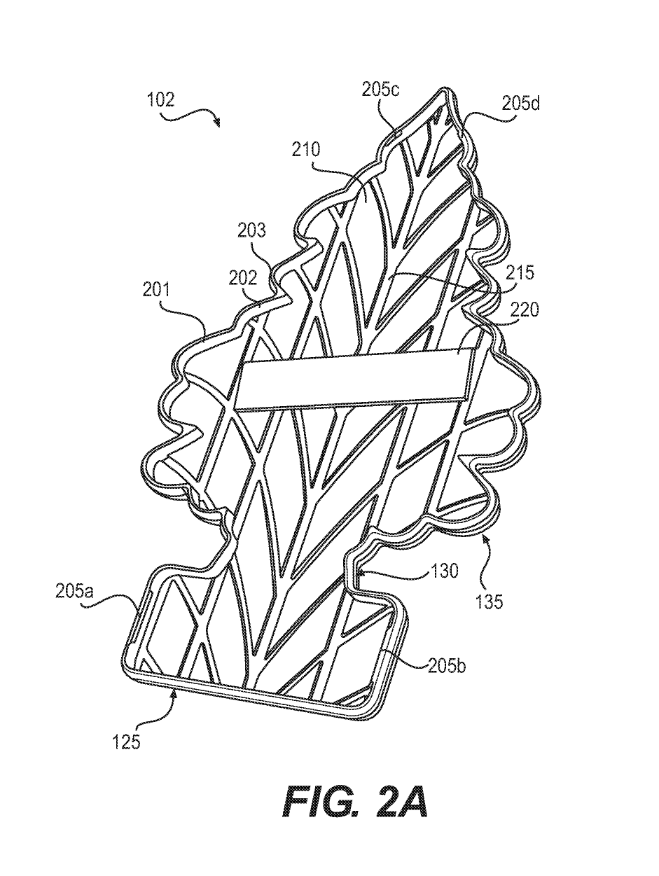

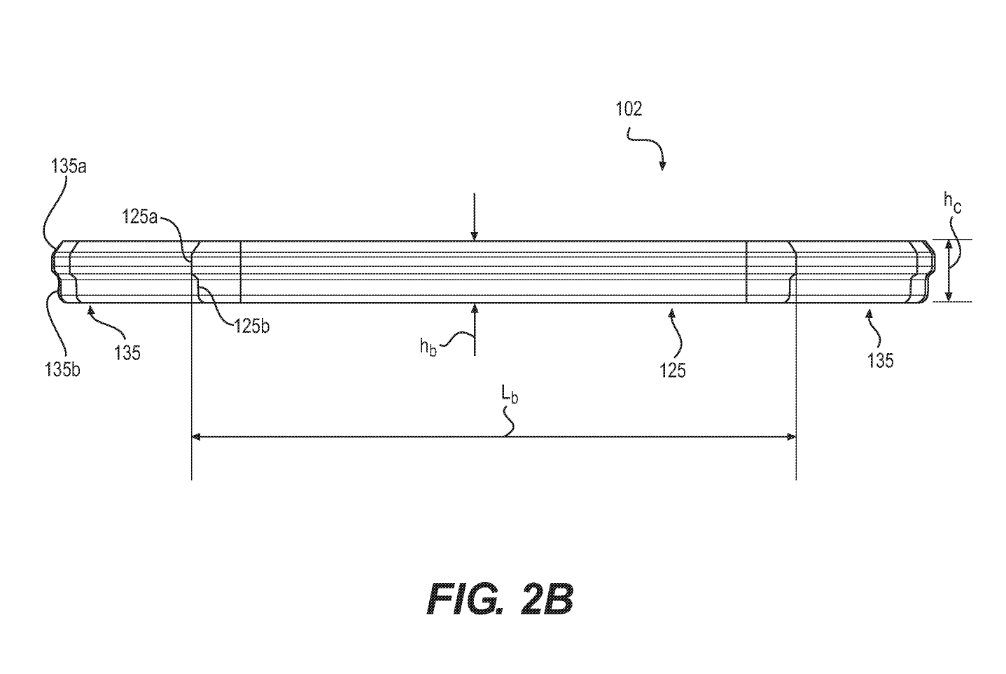

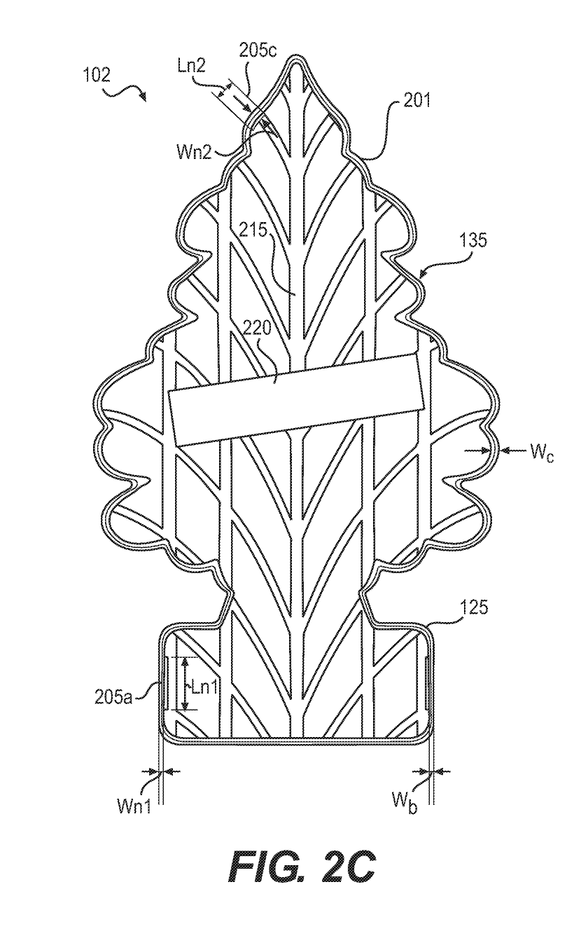

FIGS. 2A-2C illustrate, respectively, a perspective view, an end view, and a front view of the holder apparatus 102 implemented with an example holding mechanism 112. In this example, the holding mechanism 112 corresponds to or otherwise includes a holding frame 201 protruding from the front side 108 of the holding apparatus 102 and arranged to at least partially encircle the air freshener 104 (when inserted). For example, the holding frame 201 may be disposed in the bottom region 125, the trunk region 130, and the crown region 135 to completely encircle the air freshener 104 as shown in FIG. 2, or may only be disposed in the bottom region 125 to encircle just a portion of the air freshener 104, which may still provide sufficient adhesion. For illustration purposes, this design is also shown with an (optional) backing surface 210, (optional) track mark grooves 215, and an (optional) brand logo area 220, which it will be appreciated may be individually or collectively omitted and/or utilized with other variants of the holding mechanism 112 as desired.

In general, the holding frame 201 can be formed to create a pocket-like structure. In some designs, the height of the holding frame 201 may be greater than the thickness of the air freshener 104 (not shown) and the circumference of the holding frame 201 may be significantly equal to the circumference of the air freshener 104. In other designs, the height of the holding frame 201 may be less than the thickness of the air freshener 104 (e.g., when employed in conjunction with another holding mechanism 112), the circumference of the holding frame 201 may be less than the circumference of the air freshener 104, and so on.

With reference to FIG. 2B, it can be seen that in the illustrated example the bottom region 125 has a bottom height hb and a bottom length Lb, while the crown region 135 has a crown height hc. In some designs, the bottom height hb, the crown height hc, and a trunk height ht (not shown) can be significantly equal. In other designs, the bottom height hb, the crown height hc, and the trunk height ht can be different. For instance, the bottom height hb can be greater than the crown height hc. If the bottom height hb is large enough, the holder apparatus 102 can stand vertically on the bottom region 125 without additional support. This configuration may be particularly useful for decorative display purposes such that the air freshener assembly 100 may be placed on any flat surface (e.g., a table, next to a television set, on a book shelf, etc.).

In the illustrated example, the holding frame 201 has a step-like geometry with smooth edges. The step-like geometry can be divided into two parts based on their dimensions. For instance, with reference to FIG. 2B, the crown region 135 can be sub-divided into two parts 135a and 135b, and the bottom region 125 can be sub-divided into two parts 125a and 125b. The length of sub-part 125a is shown as being greater than that of sub-part 125b, thereby creating a step. The sub-parts 125b and 135b shown as having relatively smaller dimensions can act as a support for the inserted air freshener 104 (when present). Another advantage of the step-like structure is that it can prevent the inserted air freshener 104 from touching the backing surface 210. Also, if desired, an air gap can be created between the air freshener 104 and the backing surface 210.

Returning to FIG. 2A, the holding frame 201 may include an inside surface 202 and an outside surface 203, with the inside surface 202 including one or more notches 205a-205d arranged to hold the air freshener 104 within the holding frame 201. In general, the notches 205a-205d assist in holding the air freshener 104 inside the holder apparatus 102 and prevent the air freshener 104 from falling out of the holder apparatus 102. This may be especially useful when the holder apparatus 102 is oriented in a vertical or inclined position. With these notches 205a-205d, the holder apparatus 102 may even be used in a position in which the air freshener 104 would otherwise fall from the holder apparatus 102 in the absence of the notches 205a-205d, such as hanging from a ceiling or overhead surface.

In the illustrated example, the notches 205a-205d are several in number and spaced apart from each other along the inside surface 202 of the holding frame 201, with two notches 205a-205b being disposed in the bottom region 125 and two notches 205c-205d being disposed in the crown region 135. It will be appreciated, however, that the number and location of the notches 205a-205d are shown for illustration purposes only, and may vary from application to application. Further, with reference to FIG. 2C, it can be seen that the length and the width of each of the notches 205a-205d can also vary, both within and between applications. For instance, the length Ln1 of the notch 205a is shown as being greater than the length Ln2 of the notch 205c. The width of each of the notches 205a-205d is also shown as being slightly greater than the width of the holding frame 201. For instance, the width Wn1 of the notch 205a and the width Wn2 of the notch 205c is shown as being greater than the width Wb of the holding frame 201.

Turning to other aspects of the example design in FIGS. 2A-2C, the (optional) backing surface 210 may be used to protect from staining or damaging the surface of the object to which the air freshener assembly 100 will be mounted, due to the fragrance oil from the air freshener 104. The track mark grooves 215 may be used for decorative purposes and can have any pattern or shape, such as the illustrated tire tread pattern and so on. The track mark grooves 215 can also serve as air flow channels directing the fragrance when a hole is punched in the backing 210 using one or more knock-out openings (described in more detail below). The (optional) brand logo area 220 may be used for a brand logo or other text.

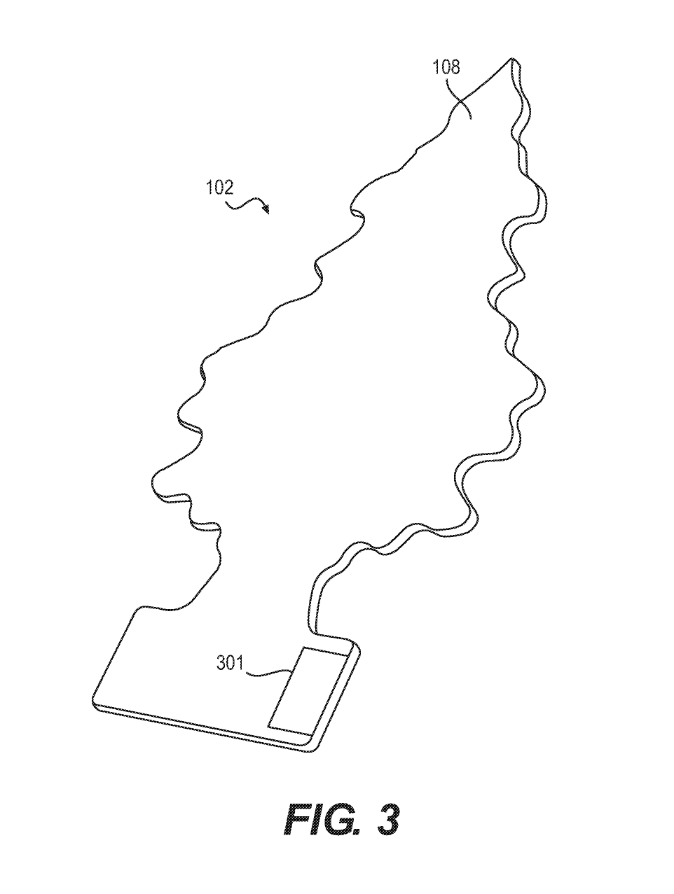

FIG. 3 is a front perspective view of the holder apparatus 102 implemented with another example holding mechanism 112. In this example, the holding mechanism 112 corresponds to or otherwise includes a latch 301 configured to hold the air freshener 104 (when present) to the front side 108 of the base 106. The latch 301 may be hinged for opening and closing to allow for removal and placement of the air freshener 104. It will be appreciated that the particular location and arrangement of the latch 301 is shown for illustration purposes only, and that the latch 301 may be disposed in different locations and arrangements as desired.

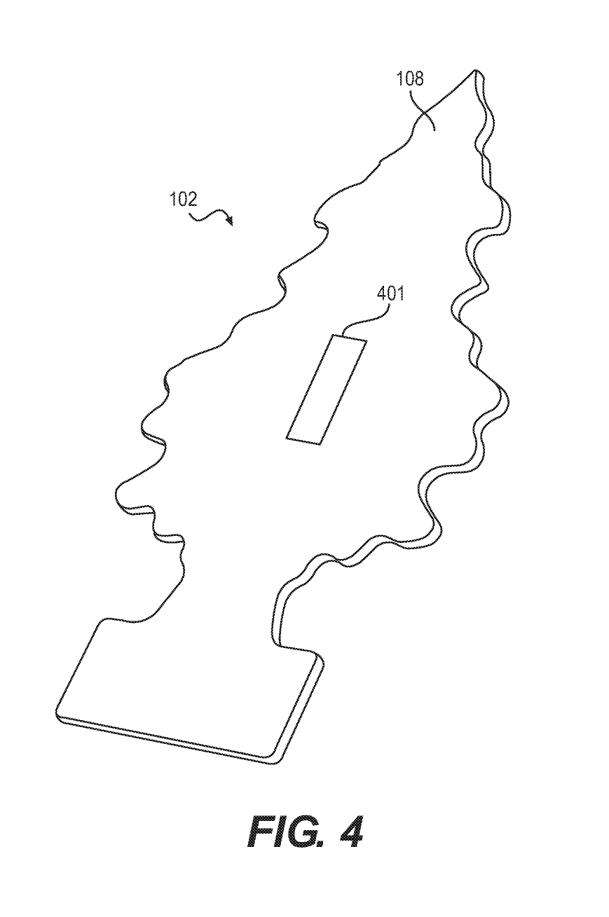

FIG. 4 is a front perspective view of the holder apparatus 102 implemented with another example holding mechanism 112. In this example, the holding mechanism 112 corresponds to or otherwise includes an adhesive strip or layer 401 configured to hold the air freshener 104 (when present) to the front side 108 of the base 106. It will be appreciated that the particular location and arrangement of the adhesive strip or layer 401 is shown for illustration purposes only, and that the adhesive strip or layer 401 may be disposed in different locations and arrangements as desired.

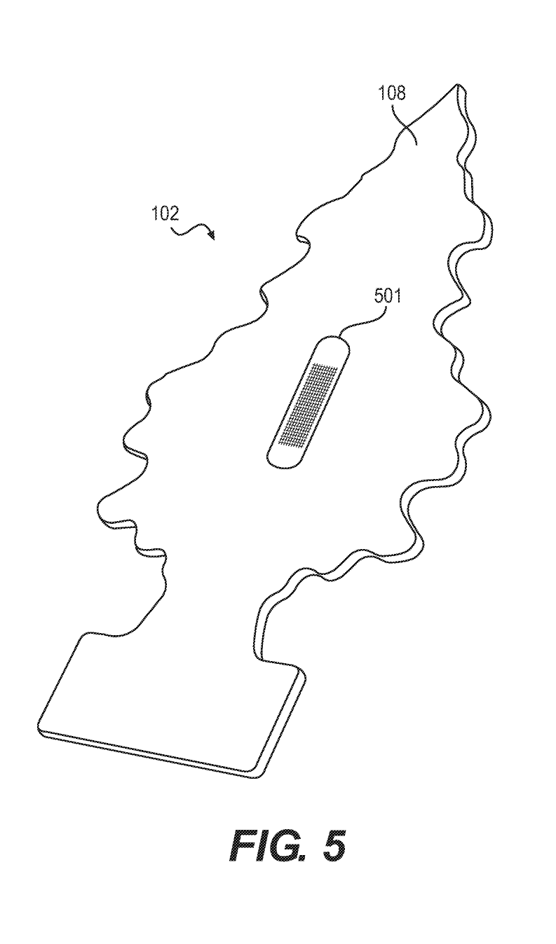

FIG. 5 is a front perspective view of the holder apparatus 102 implemented with another example holding mechanism 112. In this example, the holding mechanism 112 corresponds to or otherwise includes a hook-and-loop-based connector 501 configured to hold the air freshener 104 (when present) to the front side 108 of the base 106. It will be appreciated that the particular location and arrangement of the hook-and-loop-based connector 501 is shown for illustration purposes only, and that the hook-and-loop-based connector 501 may be disposed in different locations and arrangements as desired.

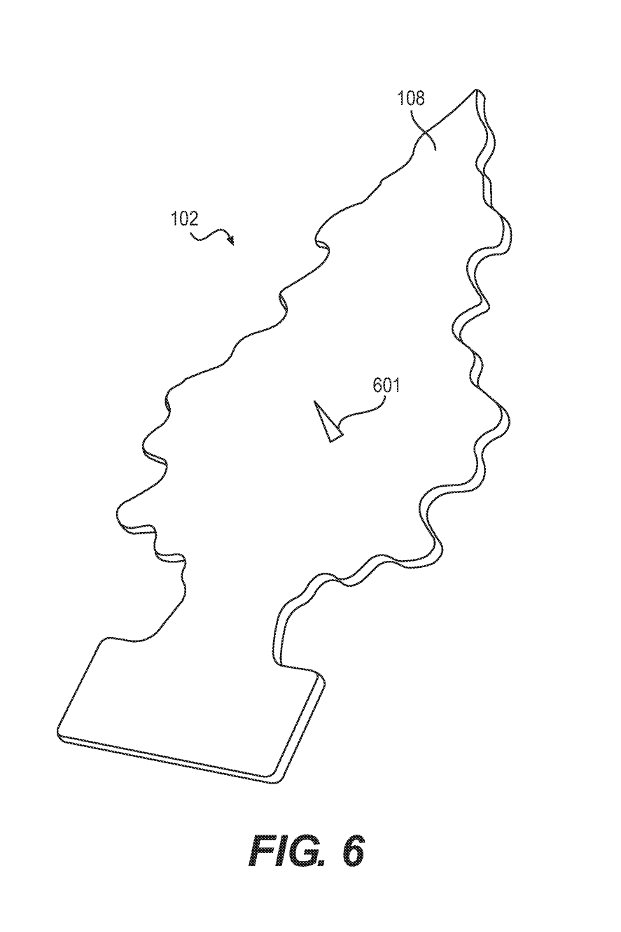

FIG. 6 is a front perspective view of the holder apparatus 102 implemented with another example holding mechanism 112. In this example, the holding mechanism 112 corresponds to or otherwise includes a tack 601 configured to hold the air freshener 104 (when present) to the front side 108 of the base 106. It will be appreciated that the particular location and arrangement of the tack 601 is shown for illustration purposes only, and that the tack 601 may be disposed in different locations and arrangements as desired.

Returning to FIG. 1, the back-side mounting mechanism 114 may also be implemented in a variety of ways. For example, it may correspond to or otherwise include a skid-resistant material, a hook-and-loop-based connector, an adhesive strip or layer, a suction cup connector, a magnetic material, and so on, or any combination of such connectors.

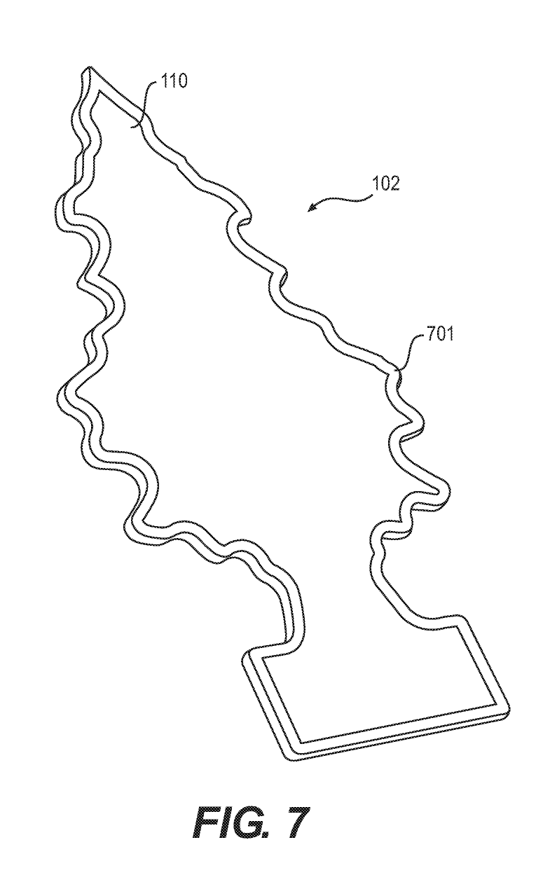

FIG. 7 is a back perspective view of the holder apparatus 102 implemented with an example mounting mechanism 114. In this example, the mounting mechanism 114 corresponds to or otherwise includes a skid-resistant material 701 arranged to mount the base 106 onto a given mounting surface (not shown). The skid-resistant material 701 may be made of a special material such as a rubber or elastomer-type material to serve as an anti-skid attachment and can be useful when the holder apparatus 102 is placed on a moving surface or a curved surface where skid resistance is desirable, such as on the dashboard of a car.

It will be appreciated that the particular location and arrangement of the skid-resistant material 701 is shown for illustration purposes only, and that the skid-resistant material 701 may be disposed in different locations and arrangements as desired. For example, the skid-resistant material 701 is shown in FIG. 7 as being arranged in a rim shape raised slightly above the back side 110 of the base 106 at its edges. However, in addition or as an alternative, the skid-resistant material 701 may instead cover a more substantial area of the back side 110 of the base 106.

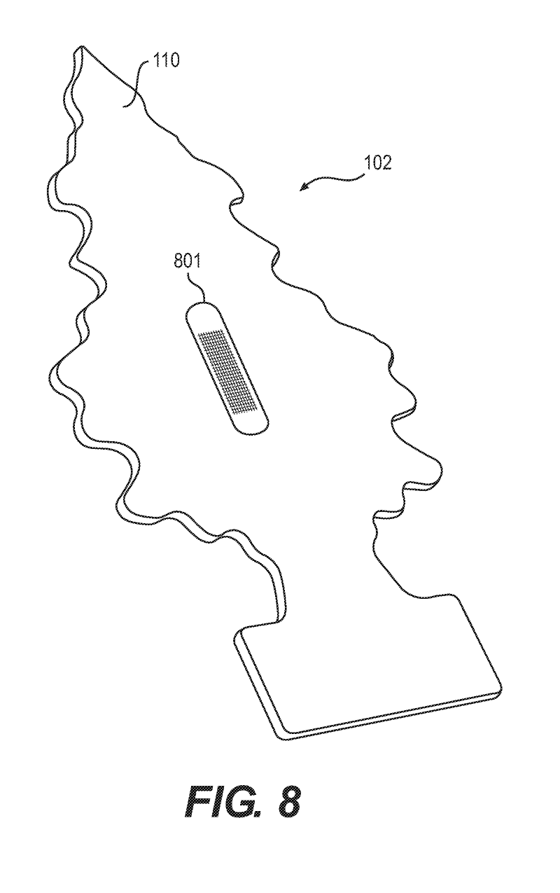

FIG. 8 is a back perspective view of the holder apparatus 102 implemented with another example mounting mechanism 114. In this example, the mounting mechanism 114 corresponds to or otherwise includes a hook-and-loop-based connector 801 arranged to mount the base 106 onto a given mounting surface (not shown). In this design, one half of a corresponding hook-and-loop combination can be attached to the back side 110 of the base 106, with the other half (not shown) being attached to the surface to which the holder apparatus 102 will be mounted. The hook-and-loop-based connector 801 can be used to attach the holder apparatus 102 to the front or rear windshield of a car, a car door, and so on. This type of mounting mechanism 114 would allow mounting of the air freshener assembly 100 on an inclined surface, a vertical surface, or even the underside of a roughly horizontal surface. It will be appreciated that the particular location and arrangement of the hook-and-loop-based connector 801 is shown for illustration purposes only, and that the hook-and-loop-based connector 801 may be disposed in different locations and arrangements as desired.

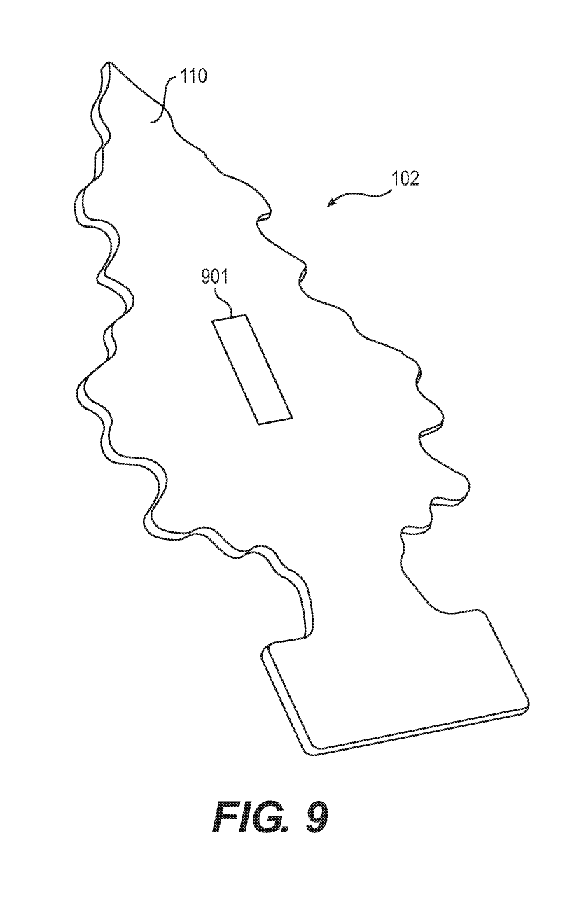

FIG. 9 is a back perspective view of the holder apparatus 102 implemented with another example mounting mechanism 114. In this example, the mounting mechanism 114 corresponds to or otherwise includes an adhesive strip or layer 901 arranged to mount the base 106 onto a given mounting surface (not shown). The adhesive strip or layer 901 can be used to attach the air freshener assembly 100 to the front or rear windshield of a car, a car door, and so on. This type of mounting mechanism 114 would again allow mounting of the holder apparatus 102 on an inclined surface, a vertical surface, or even the underside of a roughly horizontal surface. It will be appreciated that the particular location and arrangement of the adhesive strip or layer 901 is shown for illustration purposes only, and that the adhesive strip or layer 901 may be disposed in different locations and arrangements as desired.

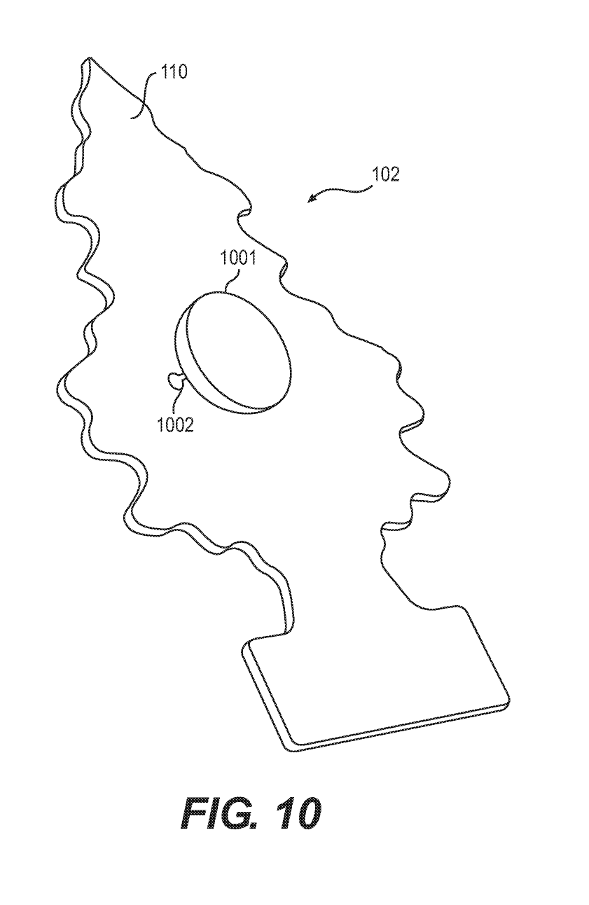

FIG. 10 is a back perspective view of the holder apparatus 102 implemented with another example mounting mechanism 114. In this example, the mounting mechanism 114 corresponds to or otherwise includes a suction cup connector 1001 arranged to mount the base 106 onto a given mounting surface (not shown). In some designs, the suction cup connector 1001 may be attached to a connector 1002, which can be separately provided. The suction cup connector 1001 may be attached to any smooth surface, such as a window or a windshield of a car, a refrigerator surface, or kitchen or bathroom wall tile, allowing the holder apparatus 102 to be mounted in any orientation on the desired surface. It will be appreciated that the particular location and arrangement of the suction cup connector 1001 is shown for illustration purposes only, and that the suction cup connector 1001 may be disposed in different locations and arrangements as desired.

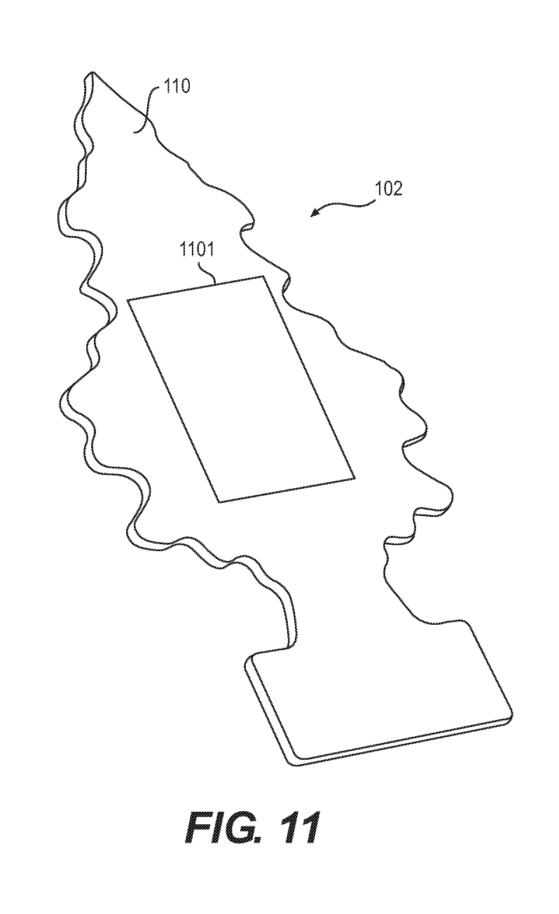

FIG. 11 is a back perspective view of the holder apparatus 102 implemented with another example mounting mechanism 114. In this example, the mounting mechanism 114 corresponds to or otherwise includes a magnetic material 1101 arranged to mount the base 106 onto a given mounting surface (not shown). For example, the magnetic material 1101 may be a ferromagnetic material or the like, which may be particularly suitable for mounting to metal surfaces such as a car door or refrigerator door. It will be appreciated that the particular location and arrangement of the magnetic material 1101 is shown for illustration purposes only, and that the magnetic material 1101 may be disposed in different locations and arrangements as desired.

As discussed above, additional air flow enhancements may be provided in some designs, including the aforementioned track mark grooves 215. In addition or as an alternative, the holder apparatus 102 may also be provisioned with one or more knock-out openings penetrating the front side 108 and back side 110 of the holder apparatus 106.

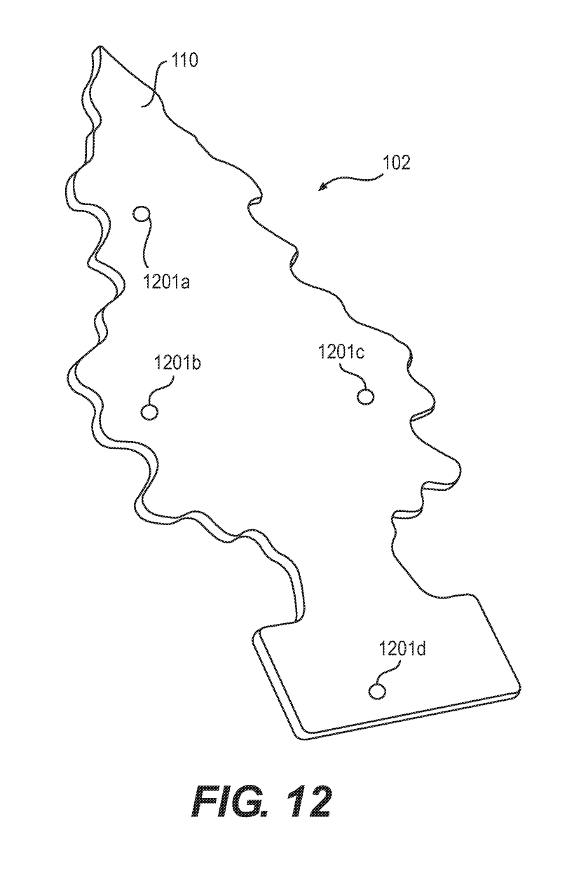

FIG. 12 is a back perspective view of the holder apparatus 102 implemented with example knock-out openings 1201a-1201d. The knock-out openings 1201a-1201d may provide additional air flow to the air freshener 104 (when inserted) and thereby increase its effectiveness. In the illustrated example, the knock-out openings 1201a-1201d are several in number and spaced apart from each other over the back side 110 of the base 106, with three knock-out openings 1201a-1201c being disposed in the crown region 135 and one knock-out opening 1201d being disposed in the bottom region 125. It will be appreciated, however, that the number and location of the knock-out openings 1201a-1201d are shown for illustration purposes only, and may vary from application to application. Further, the size of each of the knock-out openings 1201a-1201d can also vary, both within and between applications.

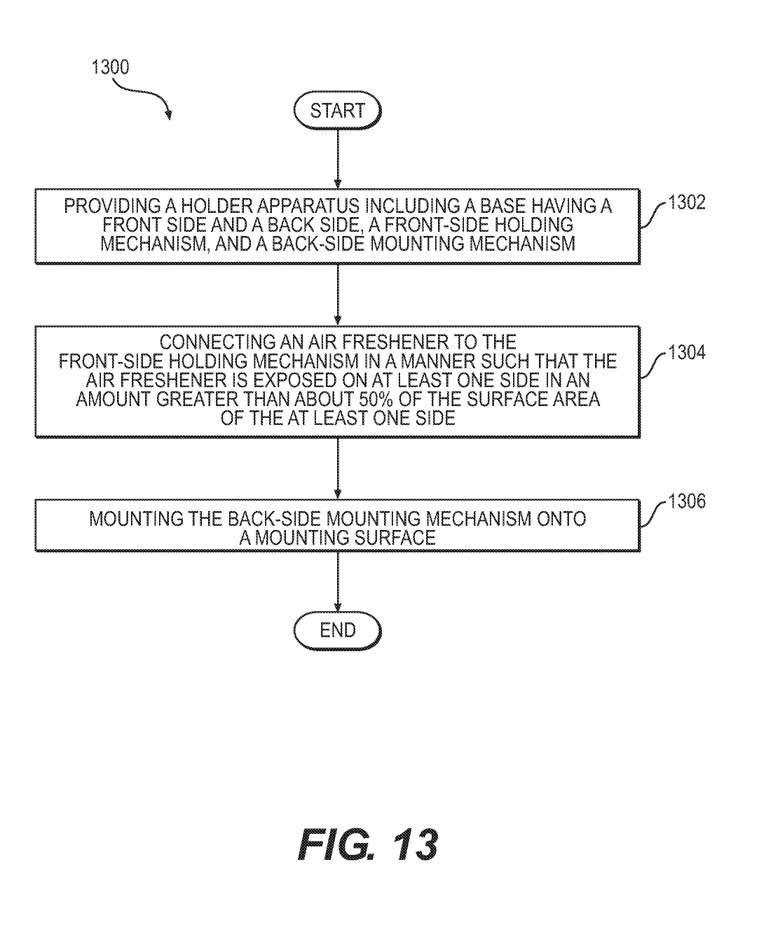

FIG. 13 is a flow diagram illustrating an example method of deploying the air freshener assembly 100. As shown the method 1300 includes providing the holder apparatus 102 including the base 106 having the front side 108 and the back side 110, the front-side holding mechanism 112, and the back-side mounting mechanism 114 (block 1302). It will be appreciated that the providing (block 1302) includes any method of obtaining or otherwise procuring the holder apparatus 102, and is not intended to be limited to the actions of any particular person or entity. The air freshener 104 may then be connected to the front-side holding mechanism 112 in a manner such that the air freshener 104 is exposed on at least one side in an amount greater than about 50% of the surface area of that side (block 1304). In either a prior or subsequent operation, the back-side mounting mechanism 114 may be mounted onto a mounting surface of the desired object (block 1306). That is, it will be appreciated that the air freshener 104 may be connected either before or after the holder apparatus 102 is mounted.

The forgoing description is provided to enable any person skilled in the art to make or use embodiments of the present invention. It will be appreciated, however, that the present invention is not limited to the particular formulations, process steps, and materials disclosed herein, as various modifications to these embodiments will be readily apparent to those skilled in the art. That is, the generic principles defined herein may be applied to other embodiments without departing from the spirit or scope of the invention.

1. A holder apparatus for air fresheners, comprising:
a base having a front side and a back side;
a front-side holding mechanism configured to hold an air freshener in a manner such that the air freshener is exposed on at least one side in an amount greater than about 50% of the surface area of the at least one side; and
a back-side mounting mechanism configured to mount the base onto a mounting surface.

2. The holder apparatus of claim 1, wherein the holding mechanism comprises a holding frame protruding from the front side of the base and arranged to at least partially encircle the air freshener.

3. The holder apparatus of claim 2, wherein the holding frame has an inside surface and an outside surface, and wherein the inside surface comprises one or more notches arranged to hold the air freshener within the holding frame.

4. The holder apparatus of claim 3, wherein the one or more notches comprises a plurality of notches spaced apart from each other along the inside surface of the holding frame.

5. The holder apparatus of claim 1, wherein the holding mechanism comprises a latch configured to hold the air freshener to the front side of the base.

6. The holder apparatus of claim 1, wherein the holding mechanism comprises an adhesive strip or layer arranged to hold the air freshener to the front side of the base.

7. The holder apparatus of claim 1, wherein the holding mechanism comprises a hook-and-loop-based connector arranged to hold the air freshener to the front side of the base.

8. The holder apparatus of claim 1, wherein the holding mechanism comprises a tack arranged to hold the air freshener to the front side of the base.

9. The holder apparatus of claim 1, wherein the mounting mechanism comprises a skid-resistant material arranged to mount the base onto the mounting surface.

10. The holder apparatus of claim 1, wherein the mounting mechanism comprises a hook-and-loop-based connector arranged to mount the base onto the mounting surface.

11. The holder apparatus of claim 1, wherein the mounting mechanism comprises an adhesive strip or layer arranged to mount the base onto the mounting surface.

12. The holder apparatus of claim 1, wherein the mounting mechanism comprises a suction cup connector arranged to mount the base onto the mounting surface.

13. The holder apparatus of claim 1, wherein the mounting mechanism comprises a magnetic material arranged to mount the base onto the mounting surface.

14. The holder apparatus of claim 1, further comprising one or more grooves recessed into the front side of the base.

15. The holder apparatus of claim 1, further comprising one or more knock-out openings penetrating the front side and back side of the base.

16. The holder apparatus of claim 1, wherein the base is made of a non-metal material.

17. The holder apparatus of claim 16, wherein the base is made of a polymeric material.

18. An air freshener assembly comprising:
the holder apparatus of claim 1; and
an air freshener held by the holder apparatus.

19. A holder apparatus for air fresheners, comprising:
a base having a front side and a back side;
front-side holding means for holding an air freshener in a manner such that the air freshener is exposed on at least one side in an amount greater than about 50% of the surface area of the at least one side; and
back-side mounting means for mounting the base onto a mounting surface.

20. A method of deploying an air freshener assembly, comprising:
providing a holder apparatus including a base having a front side and a back side, a front-side holding mechanism, and a back-side mounting mechanism;
connecting an air freshener to the front-side holding mechanism in a manner such that the air freshener is exposed on at least one side in an amount greater than about 50% of the surface area of the at least one side; and
mounting the back-side mounting mechanism onto a mounting surface.
